(19) United States
(12) Patent Application Publication
(10) Pub. No.: US 2018/0256365 A1
(43) Pub. Date: Sep. 13, 2018

(54) Stimulus Transmission and Remote Sensing System

(71) Applicant: Edward J. McCarron, Evanston, IL (US)

(72) Inventor: Edward J. McCarron, Evanston, IL (US)

(21) Appl. No.: 15/723,794

(22) Filed: Oct. 3, 2017

Related U.S. Application Data
(60) Provisional application No. 62/470,543, filed on Mar. 13, 2017.

(51) Int. Cl. A61F 2/58; A61F 2/68; A61F 2/80

(52) U.S. Cl. CPC A61F 2/583; A61F 2/586; A61F 2002/543; A61F 2/80; A61F 2/68

(57) ABSTRACT

A closed hydraulic system for transmitting a compressive force from a distal site of origin to a proximal site is disclosed. The system having a distal balloon located at the distal site, a distal transmission tube connected to the distal balloon, an exchanger connected to one end of the distal transmission tube, a proximal transmission tube connected to one end of the exchanger, and a proximal balloon connected to one end of the proximal transmission tube; the distal balloon, distal transmission tube, exchanger, proximal transmission tube and the proximal balloon, which is installed in a suitable confined space on a person and hydrostatically connected such that when the compressive force is applied to the distal balloon at the distal site, the compressive force is conveyed to the proximal balloon at the proximal site stimulating a proportional pressure sensation there in the user.

This application claims the benefit of provisional application No. 62/470,543 filed 13 March, 2017.

### FIELD OF THE INVENTION

The present invention generally relates to prostheses and ergonomic remote sensing systems.

### BACKGROUND

The absence of distal sensation is a major limitation to function of the upper extremities, in particular, whether it is due to neurological impairment or to amputation. A prosthetic socket covers part of the residual limb that still has normal sensation, while it is providing an anchor for replacement of motor function with a mechanical terminal device or hand. Voluntary closing devices permit the amputee to vary force, but with limited appreciation as to whether it is too much or too little to apply and sustain appropriate pinch or grasp. “The loss of sensation in the upper limb by amputation is the greatest factor limiting effective use of a prosthesis. A blind person cannot use an upper extremity functional prosthesis because he must rely on (visual) sensory feedback to use the hand,” Absence of sensation during the function of upper extremity prosthetic devices has persisted throughout their long history. The same sensory deficiency, during manual activities, occurs when injury to the nerve supply of the upper extremities causes loss of motor function and sensation. Orthoses designed to provide as much alternative motor capability as possible, also lack a mechanism to provide sensory feedback, other than vision, to facilitate their function.

Many prosthetic hands are comprised, superficially, of relatively dense materials which compress and conform little when grasping or pinching an object, so the total area of the palmar surface making contact during these activities is limited. This structure tends to diminish their effectiveness. The presence of some surfaces with a limited degree of compressibility, and/or a cosmetic glove, improve this somewhat. Thus, to alleviate these and other problems, this invention enables the amputee to utilize the residual limb's pressure sense to regulate prosthetic force and function.

These problems, and others, are addressed by the present invention and discussed in greater detail below.

### BRIEF SUMMARY

Currently-available prostheses lack many of the proposed features described below. With typical voluntary closing devices, the force and velocity of the motion is determined by the strength and rate of muscle activity that propels it. Feedback from a sensory transmitter can help the users to modify both speed and force. This allows for more subtle forces such as picking up an egg or maintaining a secure grip and prevent dropping.

The Stimulus Transmission and Remote Sensing system of the present disclosure enables pressure being applied during activities by a distal insensitive voluntary closing prosthetic device, orthosis or tissue to be transmitted to proximal sentient areas to enable the user to sense and adjust the distal pressure to improve function. Though finger tips have more nerve endings and are more sensitive to some stimuli, typical users will not notice an appreciable difference in the pressure applied distally or proximally.

The typical components of a Stimulus Transmission and Remote Sensing (STARS) system according to this disclosure are two small balloons, the tubes of which are connected together so their lumens communicate. The system is then typically filled with a non-compressible fluid, such as water, to become “pressure transmitter”. This is similar to a hydraulic system used in automotive applications. The balloon tubes are stabilized in appropriate distal and proximal locations and when compressed distally, during functional activities, transmit that pressure to the proximal balloon which stimulates a proportional pressure sensation there. Due to this relayed signal ending at sentient tissue, it enables improved performance. Other components of the system are variable and are disclosed herein. Such variable components relate to how the balloons are connected and the methods and components used to fill the system with fluid and remove air, the compressibility of which would reduce the effectiveness of force transmission, etc. Thus, the ability to both sense and directly vary force application provides for a higher level of performances that is closer to the normal operation of a human limb.

Functionally, the system utilizes Pascal's Law which states that pressure applied to an enclosed fluid is transmitted equally in all directions. A compressive pressure applied to one balloon will cause expansion of the other. Of course, variables can change the idealized version of the law (typically due to fluid compression and viscosity), however such variables are minimized in this design. Thus, if a distal balloon is attached to the inner surface of a prosthetic terminal device or within a glove over the thumb or index finger of a prosthetic hand, for example, and the proximal balloon is located on the inner wall of a prosthetic socket over normal skin, such as the sensitive inner surface of the forearm, then a pressure transmitting and pressure sensation stimulating mechanism has been created. A user can be trained to recognize and associate the stimuli on his proximal surface with pressures applied on the distal prostheses.

One version of the Stimulus Transmission and Remote Sensing system utilized two separate balloon tubes connected by a three-way stop cock to fill the system with water and evacuate air. Other, several modifications to such a stop cock are also disclosed to make it more compact and suitable for function in this situation. To improve the evacuation of air while it is replaced with water, a central transparent tube-like Exchange Unit was developed in conjunction with the present invention. The Exchange Unit links together two separate balloon tubes by having a male connector on each of its ends link with a female connector in the tube from each balloon. All components form a closed System filled with a non-compressible fluid, such as water. Besides providing a mechanism for filling the System with water and evacuating air, the Exchange Unit enables the adjusting of pressure in, and the expansion of, each balloon as needed.

Alternative exchange mechanisms (“exchangers”) can also be formed if two separate balloon tubes are joined by a “T” or ‘Y’ shaped connection, or by one with an acute angle projection. The projecting component of each is joined to a tube which has one or two injection port(s) to fill the system with fluid and evacuate air.

An alternative exchanger can also be created if two balloons are joined continuously to each other, during their manufacture, creating a single common tube, which is then joined continuously also by a tube with a port (designated as a “one port”) or joined continuously by a “Y” shaped tube with two injection ports (designated as “V” ports) to accomplish the exchange. A separate “T” or “Y” port could also connect to a One Port to facilitate the replacement of air by fluid. Two injection ports can also originate directly from the common tube. Other exchanger alternatives are to have the common tube joined directly by two short tubes, each of which can link with a syringe to receive an injection of fluid while the other releases air. Each short tube has a screw cap. These two continuous tubes are separated longitudinally from each other. In one case the two tubes are located on opposite sides of the common tube and this exchanger is designated as a “180 degree offset”. In the second case, the two tubes are located 90 degrees “from each other and the exchanger is designated as a “90 degree offset”.

The advantages of such an application become clear when one is experienced in prostheses and related fields. Typical devices currently on the market do not have the confluence and plethora of features contemplated and described herein.

In a first embodiment an apparatus is disclosed, the apparatus being a prosthesis having a socket, a base, and at least one voluntary closing device; and at least one hydraulic signaling system each hydraulic signaling system associated with one voluntary closing device of the prosthetic, the system having a distal balloon located on or near said voluntary closing device, a proximal balloon located in the socket of the prosthesis, and transmission tubes intermediate the proximal and distal balloons and forming a closed hydraulic system such that a compressive force on the distal balloon is transmitted to the user at the proximal balloon.

In another embodiment a closed hydraulic system for transmitting a compressive force from a distal site of origin to a proximal site is disclosed, that system having a distal balloon located at the distal site, a distal transmission tube connected to the distal balloon, an exchanger connected to one end of the distal transmission tube, a proximal transmission tube connected to one end of the exchanger, and a proximal balloon connected to one end of the proximal transmission tube, the distal balloon, distal transmission tube, exchanger, proximal transmission tube, and proximal balloon hydrostatically connected such that when the compressive force is applied to the distal balloon at the distal site, the compressive force is conveyed to the proximal balloon at the proximal site.

Such embodiments do not represent the full scope of the invention. Reference is made therefore to the claims herein for interpreting the full scope of the invention. Other objects of the present invention, as well as particular features, elements, and advantages thereof, will be elucidated or become apparent from, the following description and the accompanying drawing figures.

### DESCRIPTION OF THE DRAWINGS

The present invention may be better understood, and its numerous objects, features, and advantages made apparent to those skilled in the art by referencing the accompanying drawings.

FIG. 1 is a side on view of a spherical gate exchange unit according to the present disclosure.

FIG. 2 is a top view of the exchange unit according to FIG. 1.

FIG. 3 is a side on view of a wheel gate exchange unit according to the present invention.

FIG. 4 is a top view of the exchange unit according to FIG. 3.

FIG. 5 is end view of a male connector according to the present invention.

FIG. 6 is a cross-sectional view of a male connector according to the present invention.

FIG. 7 is a frontal view of a stimulus transmission and remote sensing unit installed on a trans radial prosthesis according to the present disclosure.

FIG. 8 is a side-view of a T-exchanger according to the present disclosure.

FIG. 9 is a side-view of a 1-exchanger with a Y-tube and two ports according to the present disclosure.

FIG. 10 is a side-view of a Y-exchanger according to the present disclosure.

FIG. 11 is a side-view of a Y-exchanger with a Y-tube and two ports according to the present disclosure.

FIG. 12 is a side-view of an acute-exchanger with one port according to the present disclosure.

FIG. 13 is a side-view of an acute-exchanger with a Y-tube and two ports according to the present disclosure.

FIG. 14 is a side on view of a continuous Y-port according to the present disclosure.

FIG. 15 is a side on view of a continuous single or one-port according to the present disclosure.

FIG. 16 is a side on view of a continuous 2-port with 180 degrees offset according to the present disclosure.

FIG. 17 is a side on view of a continuous 2-port with 90 degrees offset according to the present disclosure.

### DETAILED DESCRIPTION

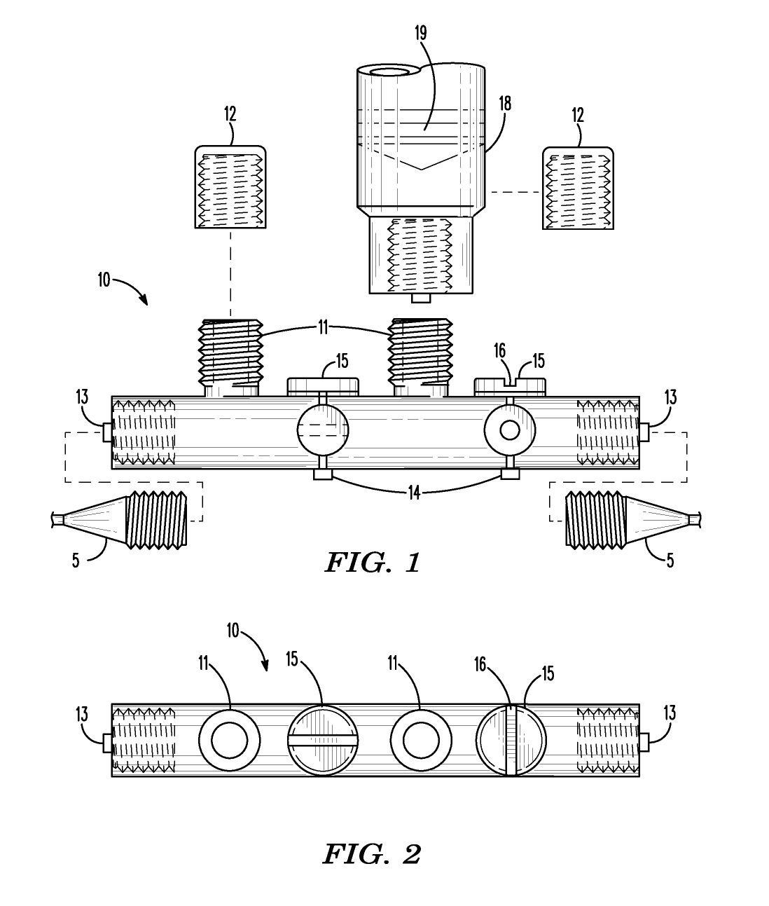

Referring now the drawings with more specificity, the present invention essentially discloses an apparatus for sensing information on and controlling a prosthesis. The preferred embodiments of the present invention will now be described with reference to FIGS. 1-17 of the drawings. Variations and embodiments contained herein will become apparent in light of the following descriptions.

Looking now to FIGS. 1 & 2 a spherical gate exchanger unit 10 is shown. A typical gate exchanger 10 is comprised of e-ports 11, which can be mated to screw caps 12, threaded connectors 13, gate spindles 14, gate controls 15, and preferably grooves 16 located on the gate controls 15. One or more e-ports 11, may be mated with a syringe 18 which will comprise at least a plunger 19. As may be clear, matable threaded attachments 5 can be screwed onto the thread connectors 13 for filling balloons or other attachments.

The exchange unit 10 disclosed in FIGS. 1-2 is designed to be compact and to make evacuation of air easier and more effective than with a standard three way stop cock. These units simplify that operation and optimize function of the system. The unit 10 is a transparent cylinder about a half inch in diameter and, by itself, two and a half inches in length. It contains, from a superior view of its upper surface, from left to right: a male connector 13, an entry-exit (e) port 11, an external gate control knob 15 with its underlying flow control gate, a second e-port 11, a second gate control knob 15 with its own underlying flow control gate and a second male connector 13. Each e-port 11 has a screw top closure in place. Each gate control 15 knob has a longitudinal groove 16 on its surface, the direction of which is the same as that of its underlying gate. When the groove runs parallel to the long axis of the exchange unit 10, then the underlying gate is open, allowing free flow of fluid. When the groove 16 is turned at right angles to the long axis of the exchange unit 10, then the underlying gate 14 is closed, blocking flow, The internal gate itself may consist of a rotating sphere with a single central tubular passage from one side of the globe to the other to allow optimal flow of fluid when aligned parallel to the long axis of the exchange unit 10.

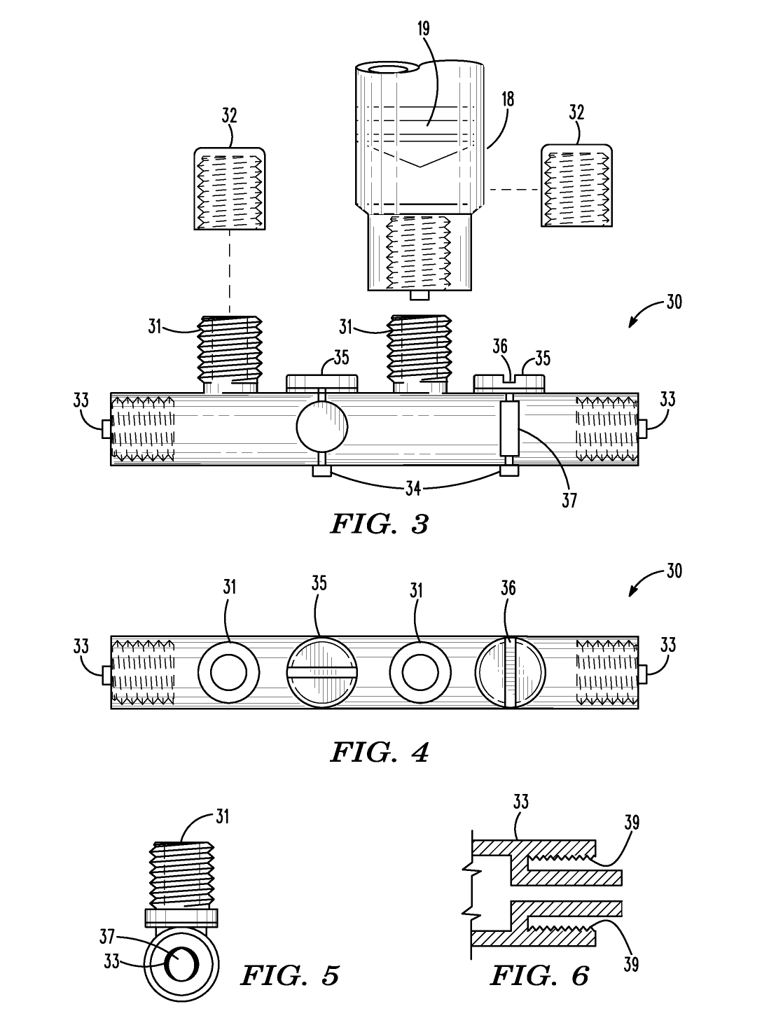

Looking now to FIGS. 3-6 a wheel gate exchanger unit 30 is shown. A typical gate exchanger 30 is comprised of e-ports 31, which can be mated to screw caps 32, threaded connectors 33, gate spindles 34, gate controls 35, and preferably grooves 36 located on the gate controls 35. One or more e-ports 31, may be mated with a syringe 18 which will comprise at least a plunger 19. As can be seen in FIGS. 5 & 6, an end view (FIG. 5) of the male connector 33 shows wheel gate 37 partially closed. A mid-line view of the male connector 33 (FIG. 6) shows an embodiment of the grooves 39 of the connectors.

The alternative gate 30 is a wheel rotating around a vertical axis and attired with an outer rubber casing layer to optimize sealing and blockage of flow or to allow flow when rotated parallel to the units 30 long axis. The rubber casing can be slightly rounded on its outer surface to make rotation of the wheel easier and avoid possible displacement. Along the casing's inner circumference, runs a low linear mid-line peaked elevation which fits into a corresponding linear trough-like depression around the rim of the wheel. This interconnection also prevents displacement of the casing during rotation of the wheel gate within the unit 30.

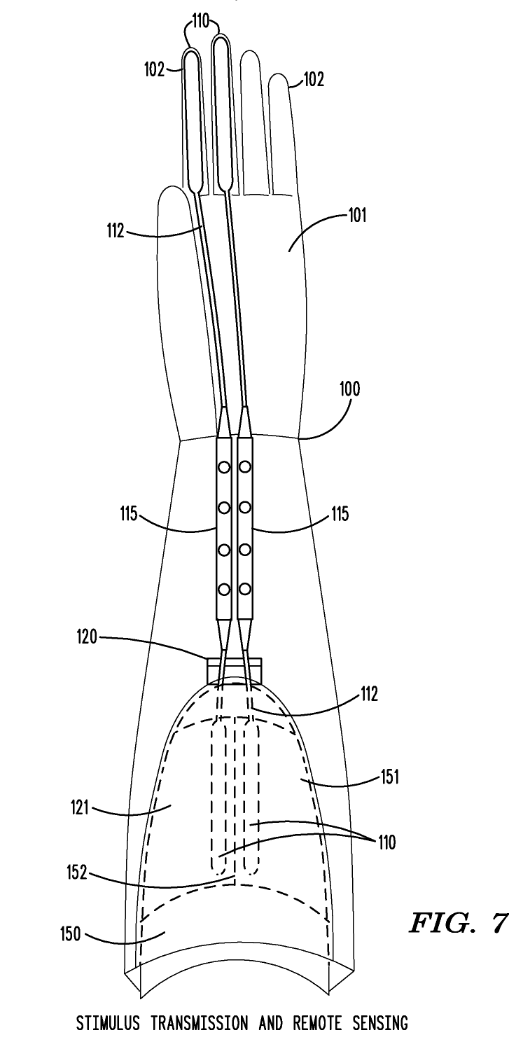

FIG. 7 shows an embodiment of a prosthetic limb 100 according to the present disclosure. A typical prosthetic 100 will comprise a hand 101 which has one or more movable appendages 102. Appendages 102 are also know in the art as a voluntary closing device as they allow a user to intentionally grip an object using the prosthesis. The prosthetic 100 is connected to the patient at the limb 150 which is at the end where the limb has been severed (often as a result of amputation). A longitudinal balloon 110 traverses from the movable appendages 102 to the limb 150 which transmits the feeling of pressure from the appendages 102 to the residual limb. Often a cuff 151 secures the location of the proximal balloons 110 relative to the limb. The cuff 151 can have a central line of stitching 152 which separates the two proximal balloons from each other and provides some stability. Intermediate the proximal and distal sections of the balloon 110 are transmission tubes 112. Both the tubes 112 and balloons 110 are preferably filled with a non-compressible fluid such as water. To ensure proper filling of the balloons 110 and tubes 112 an exchanger unit 115 is located intermediate the proximal and distal sections of the balloons 110. Exchanger 115 permits separate injection of the fluid into each balloon tube 110, evacuation of air from the system, and independent adjustment of the pressure in each balloon to optimize the function of the prosthetic 100. The prosthetic 100 may also have an entry slot 120 that facilitates the interface between the balloons 110, tubes 112, and a removable section 121 of the prosthetic 100. Exchanger 115 may be a spherical or wheel gate exchanger (as in FIGS. 1-4) or any of another type of gate exchanger disclosed herein or known in the art.

It is preferable that the balloons 110 proximal to the limb 150 are attached beneath the wall of the prosthetic 100 or other firm surface that creates a counter pressure which limits outward expansion of the balloon 110 which would otherwise lead to dispersion of the force and it's under utilization for fully effective pressure transmission and stimulation. Such a location maximizes inward pressure and directs it against adjacent sentient skin 150. The stimulus will then be approximately equal to pressure being applied to the balloon 110 located distally (adjacent movable parts 102), depending on proximal skin sensitivity. Distally, the counter pressure is supplied by the resistance of the prosthetic finger the transmitting balloon 110 overlies. While other sites are contemplated, locations that are known to be desirable for pressure sensing balloons 110 are the tips of the thumb, the index finger, the middle finger; and the front of the thumb, index, and middle fingers.

Some other surface areas 102 can also have isolated hydraulic chambers, filled with a non-compressible fluid, foam, or a compressible gas, depending on weight considerations, and serve to optimize pressure distribution, contact area and grasp. It is preferable to ensure that the locations and volumes of such compartments do not overlap and make competitive contact during grip sufficient to increase force requirements and impede closure of the distal appendages 102. Some existing prosthetic hands could be adapted to add both approaches, either by modifying their surfaces to incorporate them or by adding a glove (cosmetic or otherwise) which contains the systems distal components either on, within or beneath it. The distribution of pressure transmitting chambers distally in the hand can be duplicated proximally to determine whether the brain can learn, as expected, with instruction, repetition and possibly some training, to identify the location of distal stimuli, create a new engram and thereby derive added benefit from further sensory information.

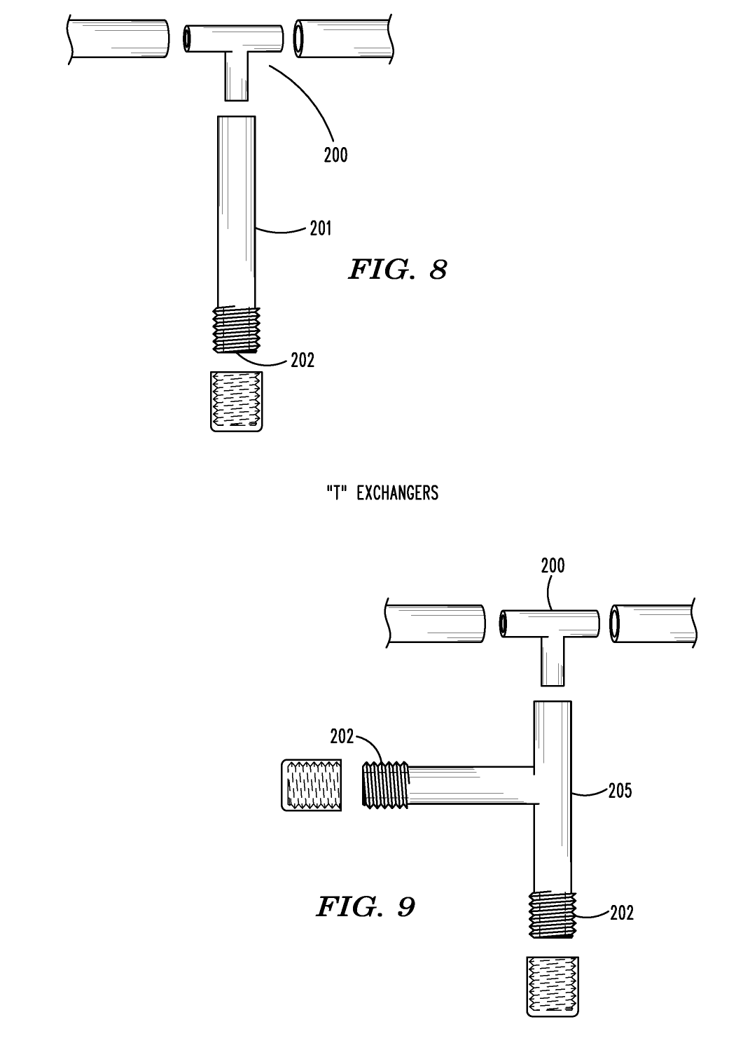

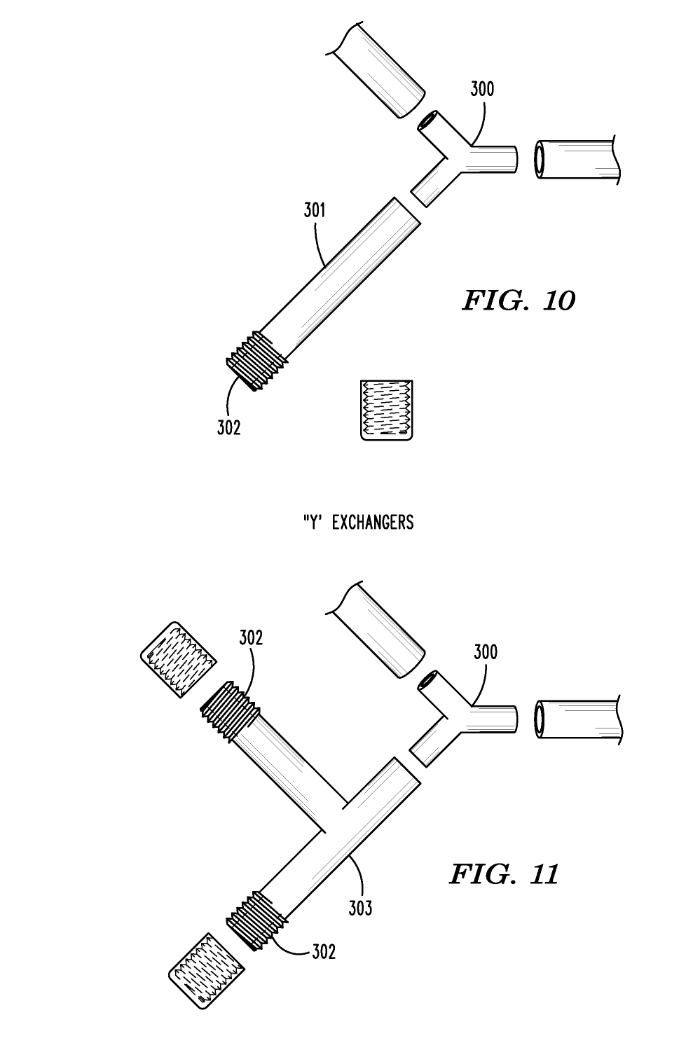

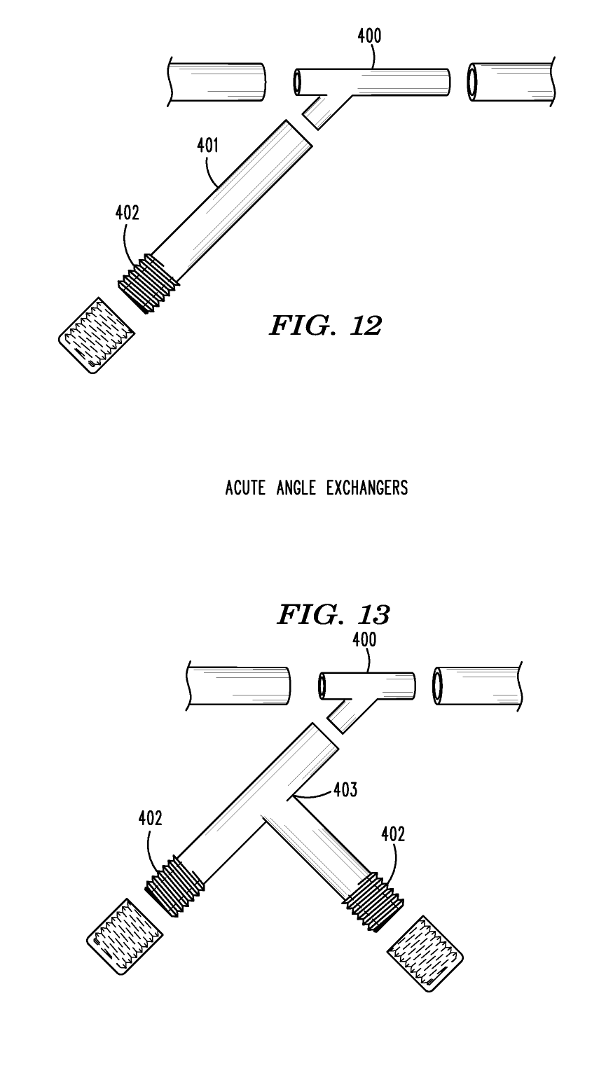

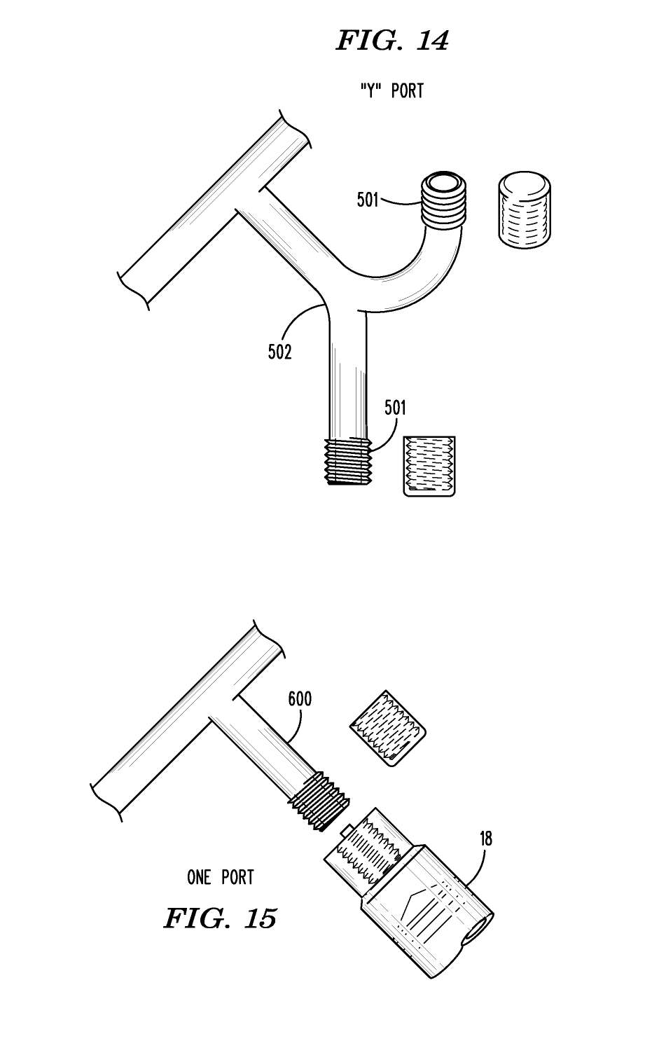

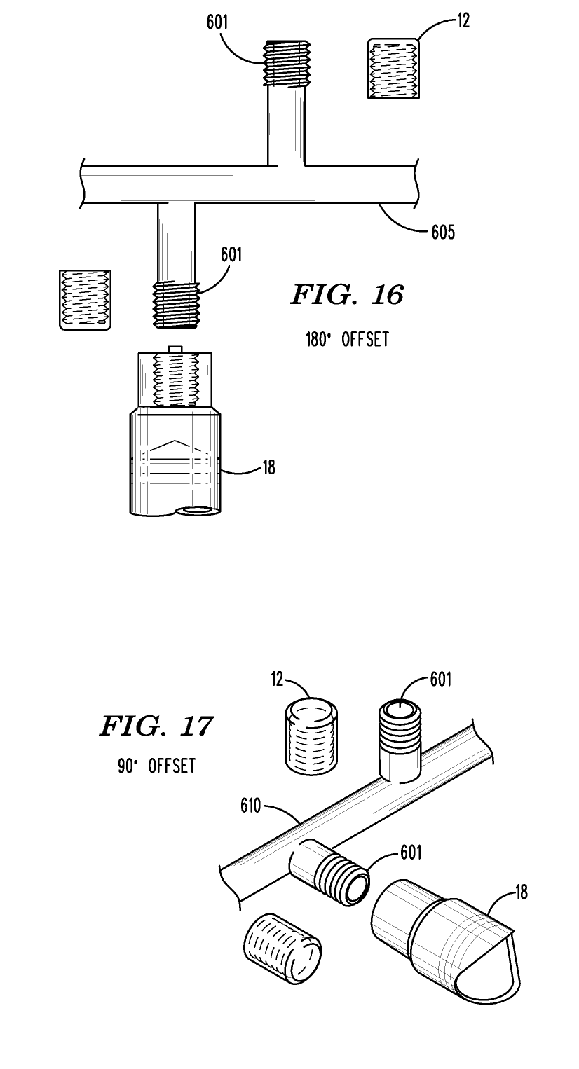

FIGS. 8-17 show additional types of exchangers or exchange units and ports that may be utilized with different prosthetics to give proper functionality of the limb. FIGS. 8 & 9 show illustrative T-exchangers 200. In FIG. 8 a simple T-exchanger 200 and tube 201 with port 202 is shown. In FIG. 9 T-exchanger 200 is paired with a Y-tube 205 having two ports 202. FIGS. 10 & 11 show illustrative Y-exchangers 300. In FIG. 10 a simple Y-exchanger 300 and tube 301 with port 302 is shown. In FIG. 11 Y-exchanger 300 is paired with a Y-tube 303 having two ports 302. FIGS. 12 & 13 show illustrative acute-angle-exchangers 400. In FIG. 12 a simple acute-exchanger 400 and tube 401 with port 402 is shown. In FIG. 13 acute-exchanger 400 is paired with a Y-tube 403 having two ports 402. FIG. 14 shows a curved Y-Port 502 with two end ports 501. FIG. 15 shows a single port exchanger 600 which may be paired with a syringe 18. FIGS. 16 & 17 show various offsets that may be contemplated between ports 601 in exchange units. In a First unit 605 the ports 601 are offset by 180 degrees. In a second unit 610 the ports are offset by 90 degrees. Such angles and illustrations are illustrative in nature and do not encompass all of the possible angles and types of exchangers utilized in prostheses according to this disclosure.

### INDUSTRIAL APPLICABILITY

The prosthetic 100 as shown in FIG. 7 defines a use for two connected balloon tubes 110 for pressure transmission. This allows for the transmission of information from distal insensitive devices 102 to proximal sentient tissue 150. Water or another similar fluid fills balloons 110 and tubes 112 to transmit said pressure information to the user. Use of an exchanger 115 as disclosed above aids in the transmission of pressure.

By incorporating two entry-exit ports (as in FIGS. 1-6), an exchanger 115 facilitates the evacuation of air from the system, because as water is being injected into one port, air has a route to escape through the other, which is then capped when air evacuation is complete. A traditional three-way stop cock does not provide this concurrent escape site, so that any significant residual air in a water filled balloon tube must be manipulated out afterwards in a separate less satisfactory, sometimes incomplete, somewhat time consuming operation by detaching the balloon tube 110, holding it vertical and squeezing or flicking it until the air rises and escapes, at which point the balloon tube is reattached. Residual air in the stimulus transmission and remote sensing system tends to diminish its efficiency in proportion to its presence, though certainly some is tolerable if sufficient useful pressure transmission still occurs.

In isolation, when not participating in a stimulus transmission and remote sensing system, a balloon tube 110 remains, structurally, just that. When incorporated into this system 100, however, it is converted into a component of a new functional entity, a pressure transmitter.

As may be clear to those skilled in the art, an advantage of the current system is that it is capable of, relatively easily, being added to existing voluntary closing prostheses or orthosis (as at 100) to provide a sensory component to their function, without the cost of new devices. In this situation, pressure transmitter tubes (110, 112) can be attached to their external surfaces by tape, covers, sheaths, staples or other means. An exchange unit 115 or units can be attached similarly on an external surface. Entry to a prosthetic socket 121 could be accomplished by creating a wide sloped channel as at 120 through the socket wall so the tubing 112 is not bent sharply, kinked or penetrated. Another possibility, in this situation, particularly initially in existing sockets, is to create a wide loop of tubing 112 attached flatly to the outer socket wall and to enter the socket obliquely by passing over its proximal edge continuing internally and attaching the rest of the transmitter against the socket wall over sensitive tissues 150 on the inner surface of the forearm. For new sockets, the system would be accommodated in initial construction Separate pre-filled pressure transmitter units could be made available to be added to a wrist-hand orthosis either by an orthotist or by an occupational therapist within their department. These would require a proximal cuff 121 for counter force application. Transmission tubes 112 which cross the wrist in the middle of its surface minimize the necessity for extra tubular length to accommodate for wrist rotation. An even more effective alternative crossing location for this purpose would be at or near the center of the wrist, midway between its flexor and extensor surfaces which would locate the exchange unit 115 component(s) within the prosthetic socket. Internal location can be accommodated most easily within the socket of amputees with shorter trans-radial residual limbs.

In some preferred applications, exchange units 115 can be located externally just proximal to the wrist and could be contained on or within a cuff, with or without channels or clips to further stabilize them. When units 115 are located externally above the wrist, a portion of the prosthetic socket proximal to this level can be made removable to facilitate installation and access to the proximal balloon tube component either on its inner surface or within appropriately designed and located pockets in a cuff that encircles the amputee's residual limb. In most embodiments, exchange units 115 are two and a half inches in length. There is, in addition, at each end, a small central tube, which projects one sixteenth inch beyond the body of the unit 115. Each enters a female connector and transmits fluid. Because each such tube functions internally within a female connector they do not contribute to the effective length of the exchange unit 115. Balloon tube 110 connectors tend to be one inch in total length. The terminal female connector section of this is a quarter inch long. Thus, the total effective length of an Exchange Unit and its two attached balloon tube connectors being four inches in some embodiments. It is theoretically possible that this total length be shorter, if possible, to allow optimal space proximally for a display of balloon tube transmitters that matches in their size and distribution that of the origin sites of their impulses in the hand. It is also possible that a proximal array of pressure transmitters 110 need not be individually of the same size, nor the area of the same dimensions, for the brain to be able to interpret their results correctly after instruction. Of note, the length of the hand in a five feet eleven inch adult male is about seven and three quarter inches and the length of the forearm is ten inches. Thus, subtracting four inches for the length of the exchange unit 115 and two female connectors from forearm length allows about six inches of length proximally for a transmitter array of pressure sensations from a distal site.

In most embodiments the skin receptors 110 would only perceive total force and not its source. This requires training by the user on the apparatus 100, but can be quickly learned in some instances. Proximally, in an above elbow amputee, there is much more regular use of bulkier muscles as the prosthetic arm is located in space by the amputee during function. Nevertheless, inside the socket 121, possibly on the medial surface, where there is less bulky muscle activity, is still a preferable location for proximal transmitters as this location also provides the necessary counter force. It is possible that the brain can sort this out, by knowing the force it perceives against the socket 121 during arm movement and separating it from the additional force it appreciates from the transmitter during grasp. Until this is known, proximal transmitter site selection should endeavor to avoid local force production from muscle activity, as far as possible.

Accordingly, although the invention has been described by reference to certain preferred and alternative embodiments, it is not intended that the novel arrangements be limited thereby, but that modifications thereof are intended to be included as falling within the broad scope and spirit of the foregoing disclosures and the appended drawings.

I claim:

1. An apparatus comprising:
a prosthesis having a socket, a base, and at least one voluntary closing device; and
at least one hydraulic signaling system each hydraulic signaling system associated with one voluntary closing device of the prosthetic, the system having a distal balloon located on or near said voluntary closing device, a proximal balloon located in the socket of the prosthesis, and transmission tubes intermediate the proximal and distal balloons and forming a closed hydraulic system such that a compressive force on the distal balloon is transmitted to the user at the proximal balloon.

2. The apparatus of claim 1 wherein the hydraulic signaling system further comprises an exchange unit located intermediate the distal and proximal balloons for filling the system with an appropriate hydraulic fluid and removing gases from the system.

3. The system of claim 2 wherein:
the prosthesis comprises more than one voluntary closing devices and each voluntary closing device is associated with at least one hydraulic signaling system.

4. The system of claim 3 wherein:
the voluntary closing devices are fingers capable of gripping an object and the distal balloon is located on the fingertip of the fingers.

5. The system of claim 4 wherein the distal balloon is a circular balloon.

6. The system of claim 3 wherein:
the voluntary closing devices are fingers capable of gripping an object and the distal balloon is located on the front of the fingers.

7. The system of claim 6 wherein the distal balloon is a longitudinal balloon.

8. The system of claim 3 the voluntary closing devices are fingers capable of gripping an object and each of the fingers is connected to two hydraulic signaling systems, the distal balloon of a first system is located on the front of the fingers and is a longitudinal balloon, the distal balloon of a second system is located on the tip of the fingers and is a circular balloon.

9. The system of claim 3 wherein the exchanger comprises:
four threaded exchange ports; and
two rotatable fluid gates.

10. A closed hydraulic system for transmitting a compressive force from a distal site of origin to a proximal site comprising:
a distal balloon located at the distal site;
a distal transmission tube connected to the distal balloon;
an exchanger connected to one end of the distal transmission tube;
a proximal transmission tube connected to one end of the exchanger; and
a proximal balloon connected to one end of the proximal transmission tube, the distal balloon, distal transmission tube, exchanger, proximal transmission tube, and proximal balloon hydrostatically connected such that when the compressive force is applied to the distal balloon at the distal site, the compressive force is conveyed to the proximal balloon at the proximal site.

11. The system of claim 10 wherein:
the distal and proximal balloons are longitudinal balloons.

12. The system of claim 10 wherein:
the distal and proximal balloons are circular balloons.

13. The system of claim 10 wherein the system is filled with water.

14. The system of claim 10 wherein the exchanger comprises:
a tubular body;
at least three threaded ports mounted to the body; and
at least one rotatable fluid gate capable of selectively impeding fluid flow through a portion of the body.

15. The system of claim 14 wherein the exchanger comprises:
four threaded exchange ports;
at least one tubular exchanger; and
two rotatable fluid gates.

16. The system of claim 15 further comprising:
a syringe matable with one or more with the threaded exchange ports.
